(19) United States
(12) Patent Application Publication
(10) Pub. No.: US 2018/0279882 A1
(43) Pub. Date: Oct. 4, 2018

(54) Concentric Helical Coils For A Wireless Link With Biomedical Implants

(71) Applicant: Board of Trustees of Michigan State University, East Lansing, MI (US)

(72) Inventors: Andrew MASON, Portland, MI (US); Ehsan ASHOORI, Haslett, MI (US)

(21) Appl. No.: 15/941,308

(22) Filed: Mar. 30, 2018

Related U.S. Application Data
(60) Provisional application No. 62/478,714, filed on Mar. 30, 2017.

(51) Int. Cl. A61B 5/00

(52) U.S. Cl. CPC A61B 5/0031; A61F 2250/0002; A61B 2560/045

(57) ABSTRACT

A biotelemetry system comprising an implanted helical coil including at least one helical coil. The system further includes a wearable external helical coil configured to wirelessly transmit data to and receive data from the implanted coil. The wearable external helical coil is further configured to wirelessly transmit power to the implanted coil. The wearable external helical coil and the implanted coil are concentric. Additionally, the wearable external helical coil includes at least one helical coil. An implanted electronic device connected to the implanted coil using a wire.

### CROSS-REFERENCE TO RELATED APPLICATIONS

This application claims the benefit of U.S. Provisional Application No. 62/478714, filed Mar. 30, 2017. The entire disclosure of the application referenced above is incorporated by reference.

### FIELD

The present disclosure relates to charging and data transfer systems and, more particularly, to biotelemetry systems and methods.

### BACKGROUND

Biomedical implants are becoming increasingly important for health monitoring, treating many disorders and controlling prosthetics. Most applications for implantables require powering and, in many cases, communicating with the implants. Using wires to connect bioimplants to an external source is generally not a desirable option due to the risk of infection and mobility limitations. Implantable batteries have been used but are undesirable in most applications because they are relatively bulky and require additional surgeries for periodic power source replacement. Furthermore, batteries do not eliminate wires would be needed to support communication with smart/programmable bioimplants. Therefore, inductive links are becoming an increasingly popular option for powering and communicating with bioimplants.

An inductive link for bioimplants consists of a transmitter coil outside the body (also called external or primary coil) which induces voltage on a receiver coil (also called internal or secondary coil). To model a coil, a parasitic resistance is considered in series whereas a parasitic capacitance placed in parallel as in FIG. 2. In such links, power transfer efficiency is one of the most important factors because low efficiency results in more power radiation that could threaten the safety of living tissues. To maximize transfer efficiency in an inductive link, the quality factor of the coils (which is a metric to indicate how many parasitic elements are built in a nonideal coil) and the coupling between them play key roles. However, due to limited space in the body for bioimplants, the sizes of these coils are restricted, which in turn limits the quality factor and coupling between the coils. Printed spiral coils (PSCs) have become popular because their planar structure makes them suitable for this space-limited application. However, the coupling between a PSC transmitter and receiver is very sensitive to misalignment between the coils. This sensitivity can decrease the transfer efficiency of the link, especially in freely moving subjects. As a result, a PSC link will often overexpose the tissue with electromagnetic energy to compensate for low efficiency, and this low efficiency can also affect the data transfer rate in communication.

### SUMMARY

In accordance with the present invention, a biotelemetry system includes an implanted coil and an external helical coil worn by a patient. In another aspect, the implanted coil may include multiple substantially helical coils which are substantially concentric with the external helical coil. A further aspect allows the transmission of data or control instructions from the implanted to external coils or vice versa. Another aspect provides a patient-implanted coil and a helical external coil, where the external coil surrounds the implanted coil during use. A method of making and/or using concentric helical coils as a wireless link for biomedical implants is also provided.

The present system is advantageous over prior devices. For example, the present system allows for misalignment of the implanted and external coils due to the helical shape of at least the external coil. Furthermore, the present system advantageously uses higher power but in a lower power density to minimize tissue damage. Moreover, the present system is less bulky and thinner thereby being less obtrusive when implanted.

The present system beneficially has a very high quality factor and high coupling coefficient compared to conventional devices. Thus, the power transferred to the implant is not much less than the power emitted. This reduces power demands which extends the life of battery systems and is very efficient. Additional advantages and features will become evident from the following description, claims and appended drawings.

### BRIEF DESCRIPTION OF THE DRAWINGS

The present disclosure will become more fully understood from the detailed description and the accompanying drawings.

FIG. 1 is a diagrammatic view showing a collar including concentric helical coils;

FIG. 2 is a schematic diagram illustrating magnetic field lines in helical coils for wireless telemetry and equivalent circuit schematic along with the coil model;

FIG. 3 is a graphical representation of changes of a coupling coefficient as a function of a tilting angle;

FIG. 4 is a graphical representation of changes of a coupling coefficient as a function of vertical displacement of the coils;

FIG. 5 is a schematic diagram of a power transfer experiment;

FIG. 6 is a graphical representation of simulated data on a rectangular plot and Smith chart;

FIG. 7A is an electromagnetic simulation of concentric helical coils in Ansys Maxwell;

FIG. 7B is a schematic diagram of power transfer in Ansys Simplorer;

FIG. 7C is a graphical representation of power transfer efficiency versus frequency simulated in Ansys Simplorer;

FIG. 8A illustrates measured s11 and s22 on Smith chart;

FIG. 8B illustrates power transfer efficiency versus frequency along with s11 on rectangular plot;

FIG. 9 illustrates power transfer efficiency versus normalized misalignments;

FIG. 10 is a depiction of fabricated coils used for measurements;

FIG. 11A illustrates an OOK data modulator;

FIG. 11B depicts an envelope detector;

FIG. 11C illustrates an oscilloscope screen shot for data transferred over the link;

FIG. 12A illustrates an example concentric helical coil in a wearable bracelet;

FIG. 12B illustrates an example lateral view of skin including concentric helical coils;

FIG. 12C illustrates an example cross sectional view of skin including concentric helical coils;

FIG. 12D illustrates an example collar for animal research;

FIG. 12E illustrates two coils in a concentric formation;

In the drawings, reference numbers may be reused to identify similar and/or identical elements.

### DETAILED DESCRIPTION

A preferred embodiment of the present system employs concentric helical coils for a wireless link. The concentric coils are used for wireless power and data transmission in implantable neuroprosthetic devices. The transmitter coil can be implemented in any continuous circle form, such as a bracelet, an arm cuff, or a collar. It is shown that this structure produces a high coupling coefficient that is less sensitive than conventional methods to coil misalignment. Furthermore, this approach reduces expose of living tissue to electromagnetic power density and also moves the wireless link far from more sensitive tissues such as brain, enables more level of power to be safely transferred. Tests show that this approach achieves a high coupling coefficient between transmitter and receiver coils (k=0.44) which decreases no more than 19.9% with the worst case of misalignment. This high coupling along with high coil quality factors (Q=470, 195) enables high power transfer efficiency of η=93.6%. This power transfer efficiency is also very resilient to misalignment between coils, with a worst case decrease of only 9%.

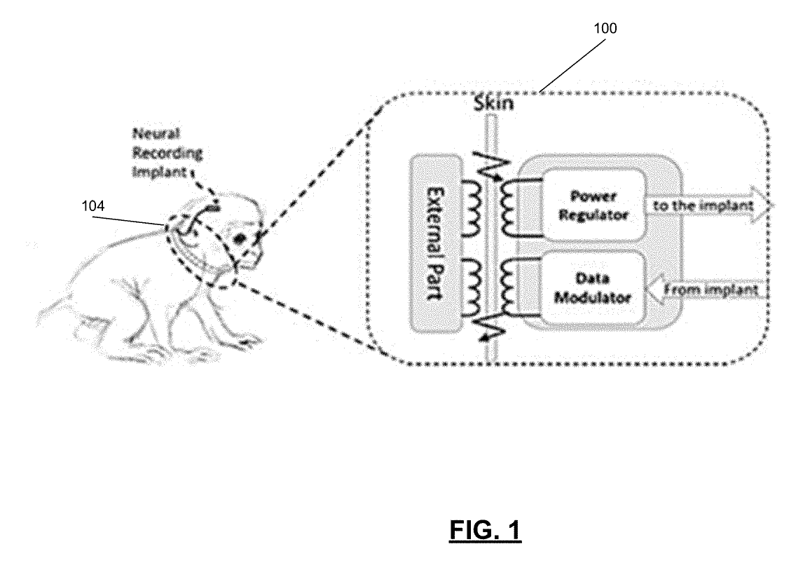

An inductive link uses the concentric helical coils for power and data transmission that is highly reliable and enables effective wireless bioimplants in freely moving subjects. The concentric helical coils provide a high coupling between coils that is also resilient to misalignment. Simulation and experiments show that a high coupling coefficient of k=0.44 could be achieved, and worst case misalignment only reduces this by less than 20%. As a result, a high power transfer efficiency of 93.6% was obtained which is also resilient to misalignment and reduces no more than 9% in the worst case of experiment. Moreover, as shown in FIG. 1, an inductive couple structure 100 can be implemented with a large cross sectional area, like concentric helical coils in an animal collar 104, allowing more power to be safely delivered while living tissue is exposed to less electromagnetic energy density. This new approach also enables separating the wireless link from the implanted electronics using under-skin wires, as shown in FIG. 1, to avoid exposing sensitive circuitry or parts of body such as the brain to electromagnetic energy.

Self-inductance, self-capacitance, and self-resonant frequency calculations in helical coils are discussed as follows. In a helical coil, self-inductance is calculated as:

$$L = μ {N}^{2} \frac{{d}^{3}}{3 {l}^{2}} ( \frac{1 - {α}^{2}}{{α}^{3}} {K}_{( α )} + \frac{2 {α}^{2} - 1}{{α}^{3}} {E}_{( α )} - 1 ) \qquad ( 1 )$$

where μ is permeability of environment, N is number of turns, d is coil diameter, l is coil length, K(α) and E(α) are the complete elliptic integrals of the first and second kind, respectively, and a is obtained from:

$$α = \frac{d}{\sqrt{{d}^{2} + {l}^{2}}} \qquad ( 2 )$$

Every passive device shows parasitic elements which restricts device performance. In helical coils, parasitic capacitance is estimated as:

$$C \approx 11.26 l + 8 d + \frac{27}{\sqrt{l / d}} \qquad ( 3 )$$

Therefore, self-resonant frequency (SRF) of the helical coil is obtained from:

SRF=½π\sqrt{LC} (4)

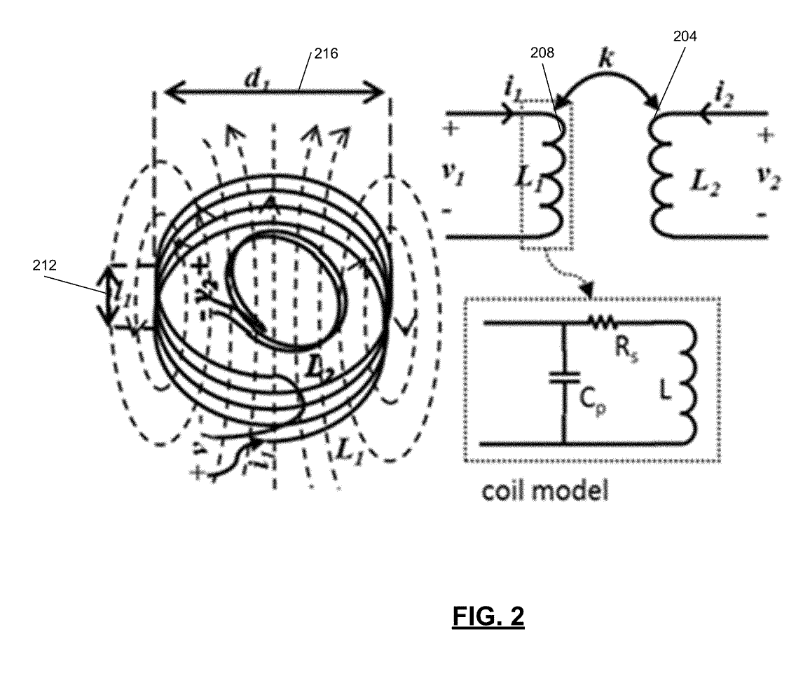

For frequencies above SRF, helical coil tends to show negative impedance and does not act as a coil anymore. Resiliency of mutual inductance in concentric helical coils is hereinafter considered. In an inductive telemetry module, mutual inductance between a pair of coils is of high importance because it directly affects the telemetry performance. FIG. 1 is a schematic diagram illustrating magnetic field lines in helical coils for wireless telemetry (left) and equivalent circuit schematic (top right) along with the coil model (bottom right). FIG. 2 illustrates the concentric helical coils where the secondary coil (L₂) 204 is implanted inside the body and primary coil (L₁) 208 is placed outside of the body. In FIG. 2, length of the primary coil is indicated with l₁ 212 and its diameter with d₁ 216. Maximizing the coupling between external and internal coils increases induced voltage on the internal coil.

Applying current i₁ in L₁ 208 generates flux ϕ that is linear proportional to i₁. If this flux changes, according to Faraday's Law, a voltage v₂ will be induced across the secondary coil (L₂) such that:

$${v}_{2 ( t )} = {n}_{2} \frac{d {φ}_{( t )}}{dt} \qquad ( 5 )$$

where n₂ is the number of turns in L₂. Considering linear relation between ϕ and i₁ we can rewrite (5) as

$${v}_{2 ( t )} = M \frac{{di}_{1 ( t )}}{dt} \qquad ( 6 )$$

where M is mutual inductance between the primary and secondary coils. In fact M is a metric that determines how strong two coils are coupled with each other. It determines how much voltage can be induced on L₂ when current changes in L₁. Similarly, we can express the voltage across the primary coil, v₁, in terms of M using:

$${v}_{1 ( t )} = M \frac{{di}_{2 ( t )}}{dt} \qquad ( 7 )$$

Mutual inductance is relative to absolute values of L₁ 208 and L₂ 204. In order to characterize the coupling between coils regardless of the coil values, coupling coefficient (k) is defined as:

k=M/\sqrt{L₁L₂} (8)

Resiliency of coils coupling coefficient to misalignment is very important due to its intense effect on efficiency of the wireless link. In concentric helical coils, misalignment can manifest as vertical displacement of the coils as well as angular misalignment or tilting. To compensate for the effect of misalignment in helical concentric coils, a proper design consists of an external coil with longer length and more number of turns compared to internal coil. This longer coil introduces more uniform magnetic field strength inside it so that coupling between the coils is more resilient to misalignment.

Quality factor and power transfer efficiency calculations are as follows. Quality factor of a coil is defined as proportion of the imaginary part to the real part of its impedance. For a coil model shown in FIG. 1, impedance is calculates as:

$$Z = \frac{{R}_{s} + j ω L}{1 - {ω}^{2} {LC}_{p} + j ω {R}_{s} {C}_{p}} \qquad ( 9 )$$

Therefore, quality factor is calculated as:

$$Q = \frac{ω L - ω ( {R}_{S}^{2} + {ω}^{2} {L}^{2} ) {C}_{p}}{{R}_{S}} \qquad ( 10 )$$

As (10) suggests, reducing Cₚ and Rₛ results in higher quality factor. It has been shown that for a helical coil made of copper, quality factor could be estimated as:

Q≈7.5dψ\sqrt{f} (11)

where ψ is an empirical parameter related to length (l), diameter (d) and spacing between turns (s).

Helical coils are not built on a substrate and it is inherent advantage for them over PSCs. PSCs are conventionally built on an FR4 substrate with relative permittivity of 4.4. This absence of substrate results in a lower parasitic capacitance in helical coils. Furthermore, using wires with bigger cross section (gauge 24 in this work) reduces parasitic resistance. Thus, higher quality factors are achievable in helical coils compared to PSCs.

Power transfer efficiency is calculated as:

$$η = \frac{{k}^{2} {Q}_{1} {Q}_{L}}{1 + {k}^{2} {Q}_{1} {Q}_{L}} \times \frac{{Q}_{2}}{{Q}_{2} + {Q}_{L}} \qquad ( 12 )$$

where Q₁ and Q₂ are the quality factors of primary and secondary coils, respectively and Q_{L} is the loaded quality factor defined as Q_{L}≈ω₀R_{L}C₂. R_{L} is the load resistance and C₂ is the tuning capacitance for receiver coil. (12) shows to achieve a high power transfer efficiency, quality factors and coupling coefficient play roles.

There are benefits of the concentric helical coils for wireless implantable devices. Modern wireless implantable devices primarily utilize wire-wound coils and PSCs for power and data transmission. Wire-wound coils with air or ferrite cores are three dimensional structures that lose efficiency as they are shrunk down to minimize the bulk that can make an implant uncomfortable to the user. PSCs are planar, two dimensional, devices that can be smaller, but they require a rigid substrate that limits the locations in which they can be comfortably implanted. In contrast, concentric helical coils have very strong coupling, allowing the implanted coil to be a simple thin wire formed in only one or two loops around, for example, the wrist, arm or neck. No bulky device or rigid substrate is required, making them more comfortable for the user. Small magnets can be used both in implant and external side to prevent the implant coil from moving.

Another concern for wireless implants is electromagnetic exposure of nearby tissue. Modern implantable coils are usually on the order of 1-2 cm in diameter to reduce their bulk. However, transferring over a small volume of the living tissue exposes it to a high electromagnetic power density which limits the maximum power that can be safely delivered to the implanted device. Furthermore, poor tolerance to misalignment between the coils can even worsen the situation because in case of misalignment, transmitted power should increase to compensate for degraded efficiency of the link. In contrast, concentric helical coils circumvent these problems. The thin wire can be implanted beneath the skin and formed with large diameter (depending the location in the body; ˜5-10 cm), thus maintaining a lower power density exposure for living tissues. Moreover, the implanted coil can be coupled with an external coil in the form of a bracelet, arm band or animal collar, forming an inductive link that is very tolerant to misalignment and eliminates the need for high power density to overcome efficiency loss to misalignment.

Using under-skin wires commonly found in existing implants like the deep brain stimulator. In bioimplant applications where enough space is not available at the location of interest like brain, under-skin wires can be used. Therefore, concentric helical coils provide the opportunity to wirelessly transfer the power in a part of the body that not only has more space but also has less sensitive tissue to temperature increase, such as the arm or wrist in contrast to brain. Hence, because concentric helical coils produce low power density exposure and can be placed in adjacent of less sensitive tissues, this wireless link can be used to transfer higher levels of power.

The present helical coils for wireless link in bioimplants. Since the power absorption in live tissue is increasing by frequency, it is generally preferred not to have very high carrier frequencies. On the other hand, recall from (10), in order to have high quality factor, we would like to have a higher carrier frequency. Carrier frequency of f=13.56 MHz is an optimum point for bioimplants application which is within the ISM band. Choosing f=13.56 MHz, an SRF≈5×f≈68 MHz is desirable for helical coils to stably operate at the desired frequency.

For the receiver (internal) coil, we started with a single turn coil to avoid discomfort for the subject as much as possible. For transmitter (external) coil, we considered longer coil with more turns in order to both having a stronger coupling as well as higher resiliency against misalignment. Since the initial incentive of this work was design of a wireless link for bioimplant used in primates, the geometry of external coil was chosen in a way to be mountable on the animal collar. Considering a medium size primate, the diameter of the coil is d₁=70 mm with the length of l₁=20 mm. Increasing number of turns of the external coil increases its self-inductance as well as coupling between the coils. However, calculations shows that for N=4 and N=5, self-resonant frequency equals SRF=63 MHz and SRF=50 MHz, respectively. Therefore, external coil was made with 4 turns. A typical skin thickness of 5 mm led to internal coil diameter of d₂=60 mm. Due to frequency limitation for increasing L₁, after initial simulations it turned out the number of turns for L₂ should increase to two in order to achieve a more strongly coupled link.

Quality factor, coupling coefficient and its resiliency to misalignment can be ascertained from simulations and expected experiments. To study the effect of misalignment on concentric helical coils, prior to fabrication, a pair of coils with the geometry obtained from calculation and presented in table I was simulated using ANSYS Electromagnetics Suite. The coupling coefficient was evaluated for various levels of vertical displacement and tilting between the coils. Furthermore, experiments were performed using fabricated coils and measured by Agilent FieldFox N9917A vector network analyzer.

Measured quality factor of the fabricated coils show Q=470 and Q=195 for external and internal coils, respectively. These values are up to an order of magnitude higher than PSCs used for wireless power transfer in this range of frequency. Furthermore, coupling coefficient obtained from this structure is k=0.44 which is a very appropriate value for an inductive link.

TABLE I
Characteristics of the Coils

| | Number | Coil | Coil | | Q @ |
|---|---|---|---|---|---|
| | of turns | diameter | length | Inductance | f = 13.56 MHz |
| External coil | 4 | 70 mm | 20 mm | 1.9 μH | 470 |
| Internal coil | 2 | 60 mm | 10 mm | 0.5 μH | 195 |

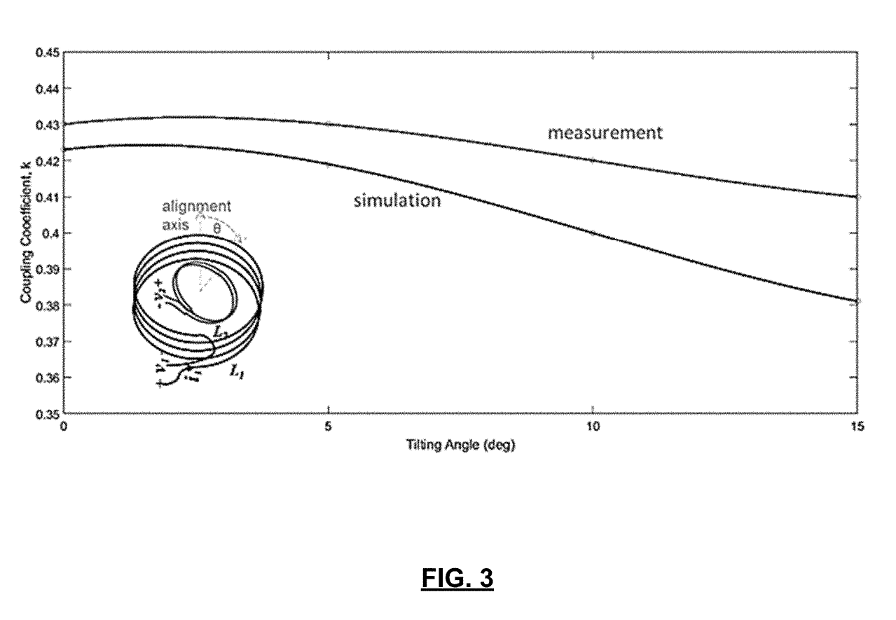

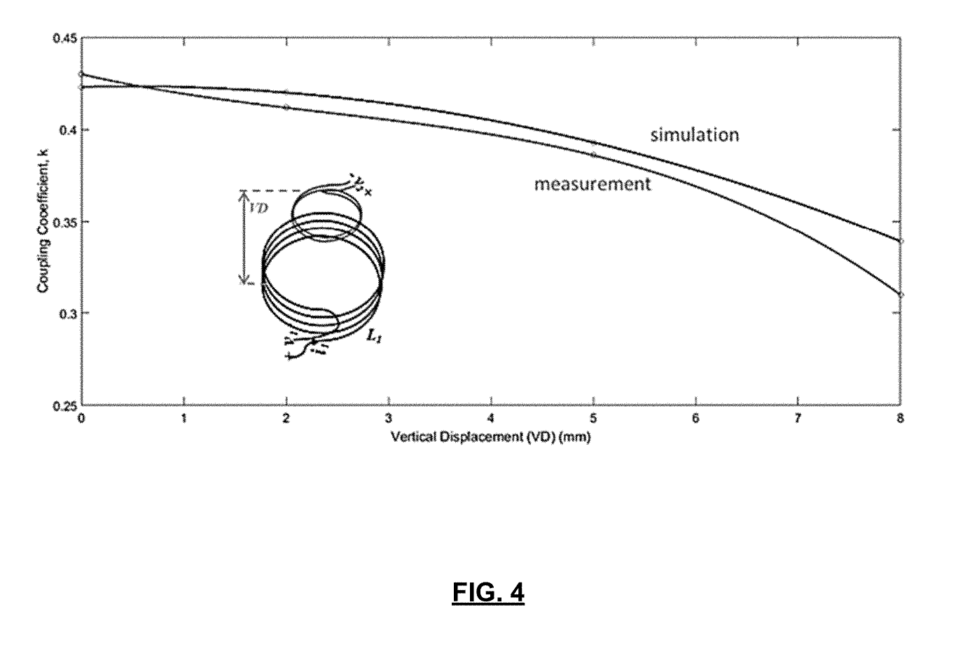

FIG. 3 is a graphical representation of changes in a coupling coefficient as a function of a tilting angle. As it is shown in FIG. 3, tilting of the external coil up to 15° can decrease the coupling coefficient no more than 9.9%. Moreover, FIG. 4 shows the change of coupling coefficient in the case of vertical displacement of the coils. It is shown that the coupling coefficient decreases by 19.9% when the displacement is 8 mm. Therefore, this resilient coupling coefficient results in a more reliable telemetry module. A performance comparison of this link with others is presented in table II.

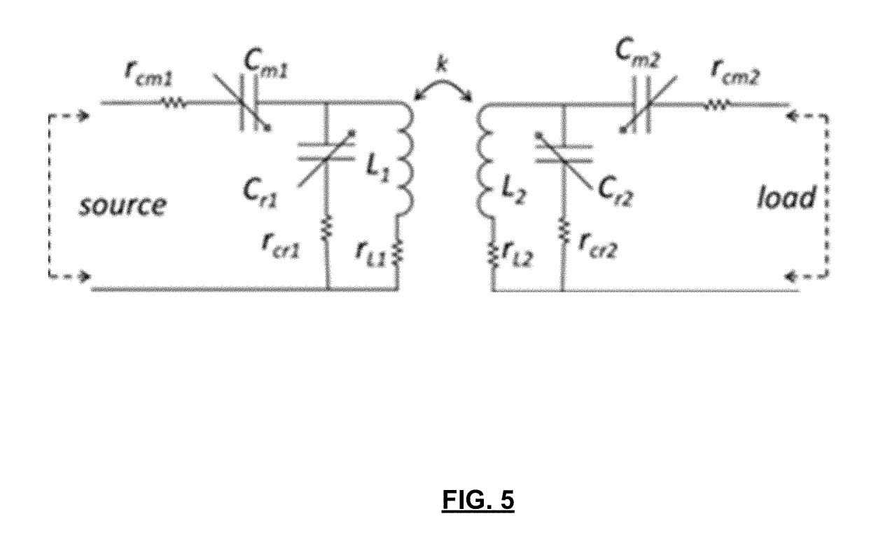

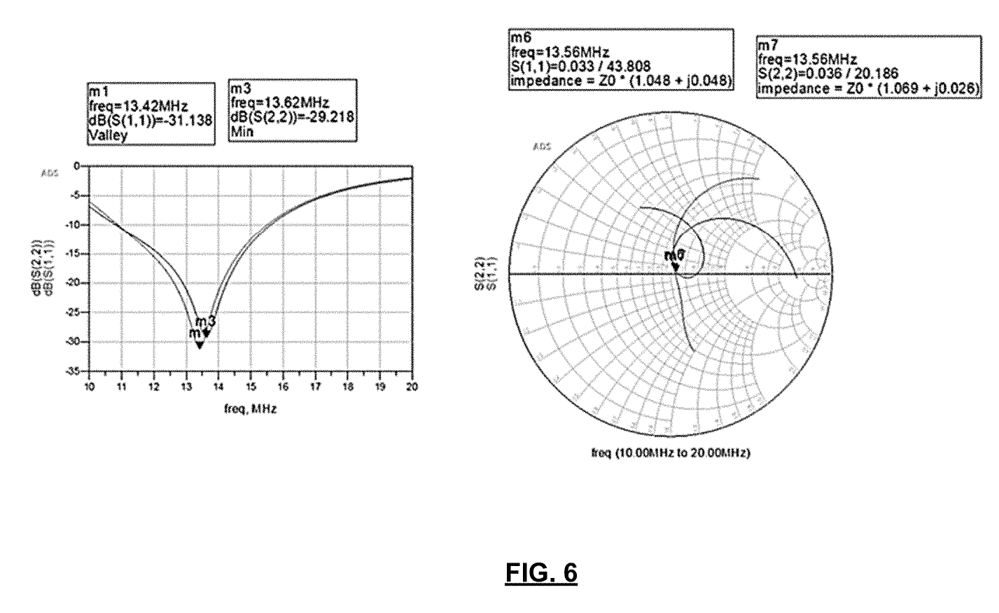

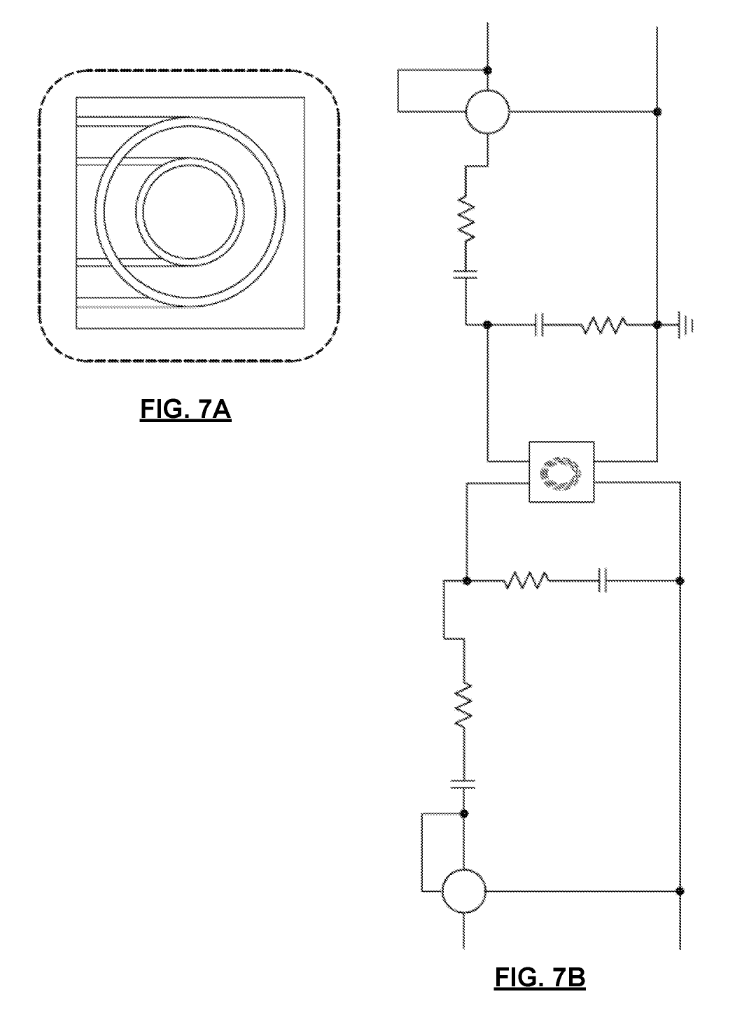

For power transfer efficiency test, a pair of LC tanks was arranged using helical coils. FIG. 5 is a circuit schematic for a power transfer experiment. In FIG. 5, L1 and L2 are helical concentric coils, Cr denotes tank capacitance and Cm employed to match the circuit with 50Ω load or source. In this setup, ceramic capacitors were used which exhibit 1-2Ω resistance at f=13.56 MHz. All known parasitic elements were added to the circuit model in Advanced Design System (ADS) to get more accurate results for tuning the circuit. It is worth mentioning that introducing matching capacitors Cm shifts the Cr value from the primarily calculated value as Cr=1/ω²L. FIG. 6 presents simulated data from ADS on a rectangular plot and Smith chart. S11 and S22 are around −30 dB which means circuit is well matched to 50Ω. Using the extracted values for capacitors from ADS with the coil model from Ansys Maxwell, a circuit model for wireless power transfer was modeled in Ansys Simplorer which shows simulated transfer efficiency of 94.8%. FIGS. 7A-C shows the simulation steps and the final result for efficiency.

FIG. 7A depicts an electromagnetic simulation of concentric helical coils in Ansys Maxwell. FIG. 7B illustrates a schematic for power transfer in Ansys Simplorer. FIG. 7C illustrates power transfer efficiency versus frequency simulated in Ansys Simplorer.

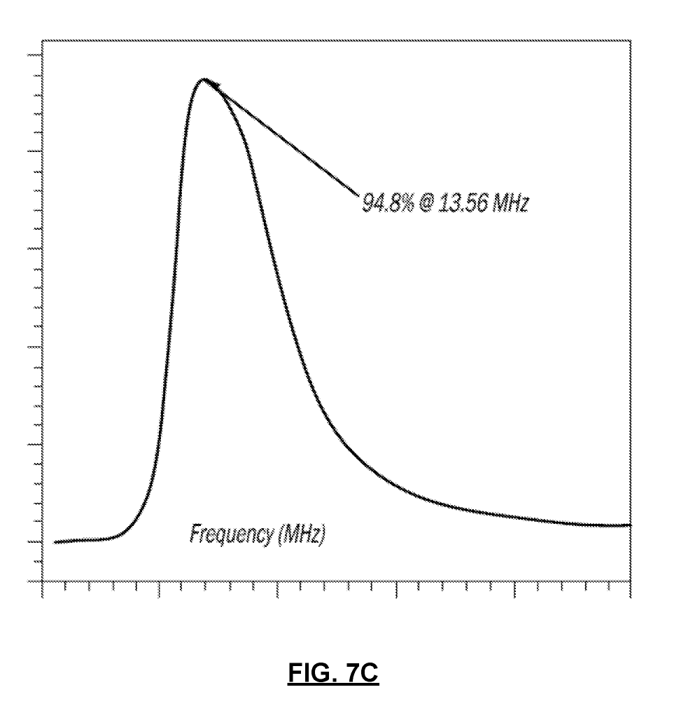

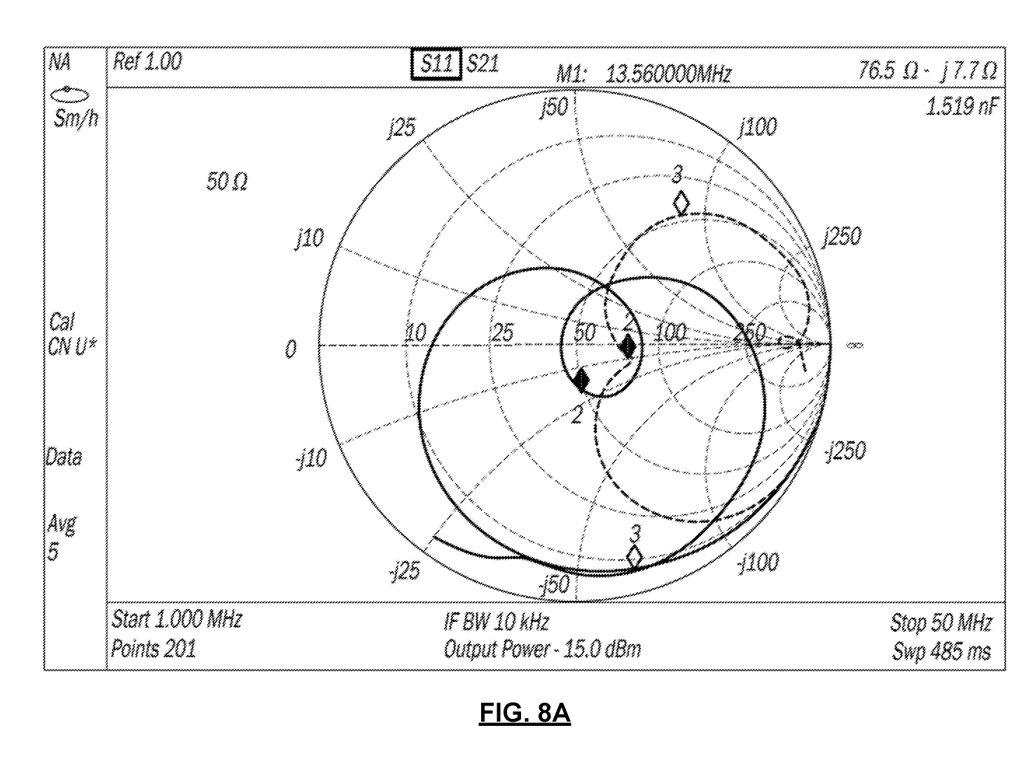

After obtaining the values for all circuit elements from the software, the circuit was implemented using fixed ceramic capacitors as well as trimmer capacitors for fine tuning the circuit. Trimmer capacitors (9702-2) from Johnson Manufacturing were used which have variable capacity between 2.5-10 pF. FIG. 8A illustrates measured s11 and s22 on Smith chart. FIG. 8B illustrates power transfer efficiency versus frequency along with s11 on rectangular plot. FIGS. 8A and 8B illustrate the measurement results for circuit matching and power transfer efficiency when frequency is swept from 1 MHz to 50 MHz. The efficiency is about 93.6% at fᵣ=13.56 MHz which is in match with simulation results from Ansys.

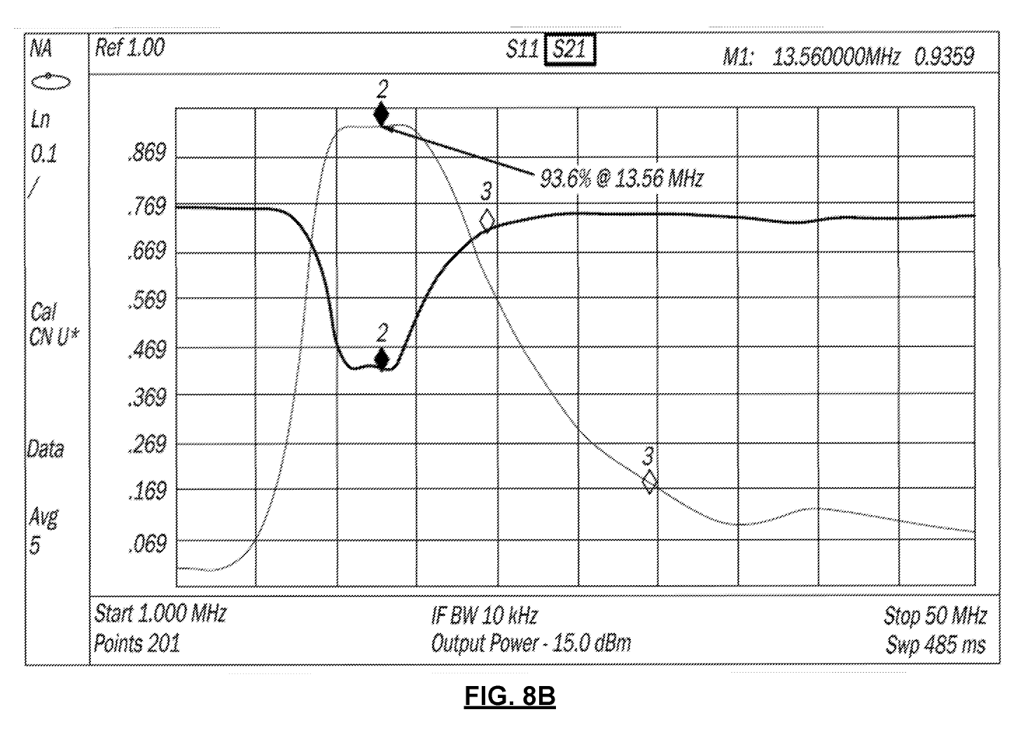

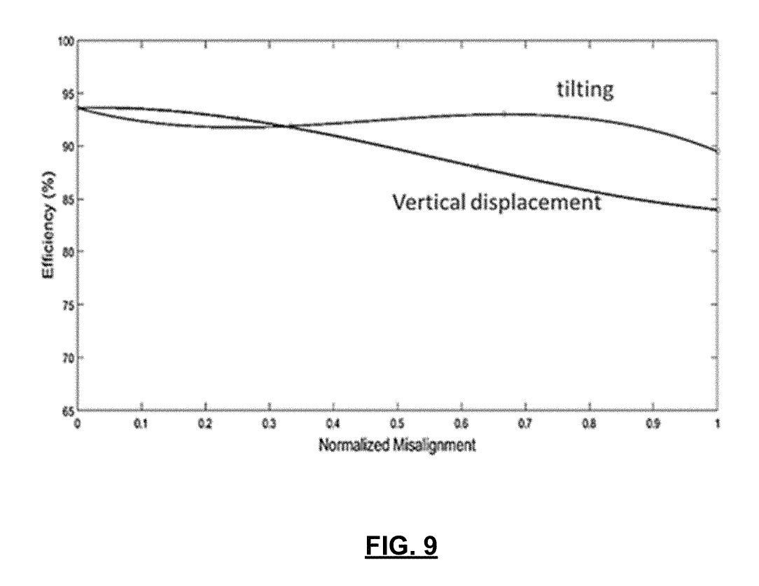

FIG. 9 illustrates power transfer efficiency versus normalized misalignments. Specifically, FIG. 9 illustrates tilting of 15 degrees and vertical displacement of 1 cm. Moreover, as depicted in FIG. 9, our experiments show that vertical and angular misalignment can decrease power transfer efficiency no more than 9% and 4%, respectively, which shows a great resiliency as expected.

FIG. 10 shows the fabricated coils used for measurements. Table II compares measured key elements of wireless link introduced in this work with others. This comparison shows a strong coupling coefficient in this structure which is also very resilient to misalignment. As a result, this link has a high and resilient power transfer efficiency which makes it very interesting for bioimplants especially in freely moving subjects.

Table II, shown below, includes a comparison of key elements of wireless link in this work with published papers. The published papers include: (i) G. Simard, M. Sawan, and D. Massicotte, “High-speed OQPSK and efficient power transfer through inductive link for biomedical implants,” IEEE Trans. Biomed. Cir. and Sys., vol. 4, no. 3, pp. 192-200, June 2010; (ii) U. M. Jow and M. Ghovanloo, “Modeling and Optimization of Printed Spiral Coils in Air, Saline, and Muscle Tissue Environments,” IEEE Trans. Biomed. Cir. and Sys., vol. 3, no. 5, pp. 339-347, October 2009; (iii) E. Ashoori, F. Asgarian, A. M. Sodagar and E. Yoon, “Design of Double Layer Printed Spiral Coils for Wirelessly-Powered Biomedical Implants,” in Proc. Eng. Med. Biol. Soc., 33rd Int. Conf. of the IEEE, 2011, pp. 2882-2885; (iv) U. Jow and M. Ghovanloo, “Optimization of data coils in a multiband wireless link for neuroprosthetic implantable devices,” IEEE Trans. Biomed Circuits Syst, vol. 4, no.5, pp. 301-310, October 2010; and (v) B. Kallel, 0. Kanoun,

TABLE II
Comparison of Key Wireless Link Parameters

| | TBioCAS | TBioCAS | EMBC | TBioCAS | IET Pow. | |
|---|---|---|---|---|---|---|
| | ′10 | ′09 | ′11 | ′10 | Elec.′16 | This work |
| structure | Planar | Planar | Planar | Planar | Planar | Helical |
| Application | Bio- | Bio- | Bio- | Bio- | Wireless | Bio- |
| | implants | implants | implants | implants | sensors | implants |
| TX/RX Size | 24/24 | 38/10 | 20/11 | 79/10 | (n × 15)/30 | 70/60 |
| (outer diameter) | | | | | | |
| [mm] | | | | | | |
| Coupling | 0.38 @ | 0.07 @ | 0.19 @ | 0.036 @ | 0.18 @ | 0.44 @ |
| coefficient | 5 mm | 10 mm | 5 mm | 10 mm | 10 mm | 5 mm |
| Δk/k due to | — | — | — | 70% | 33% | 19.9% |
| displacement | | | | | | |
| Δk/k due to | — | — | — | 9.5%, 11.6% | — | 9.9% |
| tilting | | | | | | |
| Power transfer | ~60 @ | 72.2 @ | 79.8 @ | 65 @ 5 mm, | 29.3 @ | 93.6 @ |
| efficiency [%] | 5 mm | 10 mm | 5 mm | 57 @ 10 mm | 5 cm | 5 mm |

and H. Trabelsi, “Large air gap misalignment tolerable multi-coil inductive power transfer for wireless sensors,” IET Power Electronics, vol. 9, Iss. 8, pp.1768-1774, 2016.

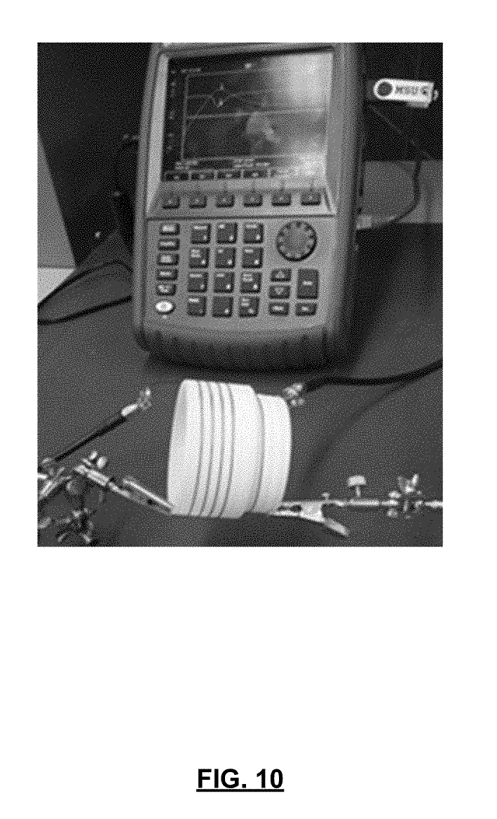

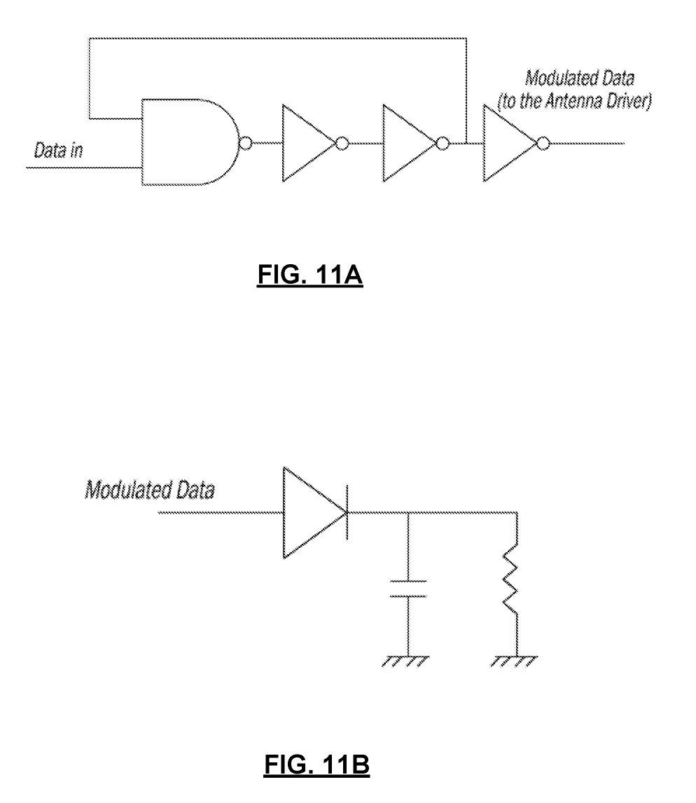

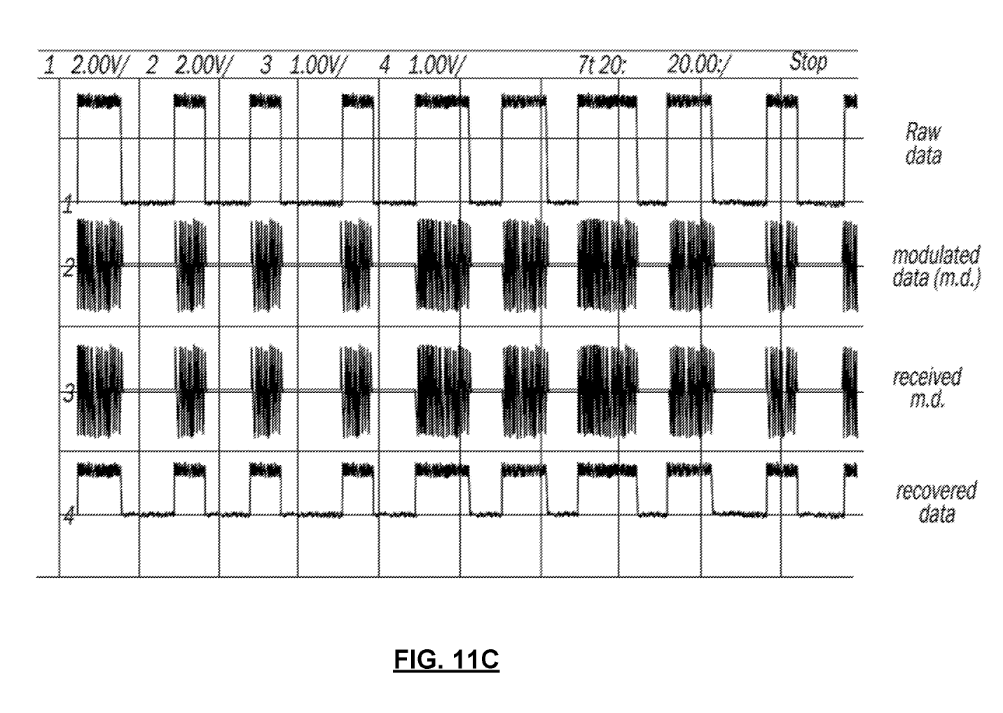

To evaluate the capacity of data rate using concentric helical coils, a backtelemetry link was set up to transfer data from implant side to the outside world. Since there is a space limit in implant applications, OOK modulation is a wise choice due to the simplicity of its design. The structures for OOK modulator and demodulator used in this work are shown in FIG. 11 along with oscilloscope screenshot of data transmission. The data transfer rate of 100 Kbps was obtained where the carrier frequency is 20 MHz. In order to get higher data rate faster digital gates can be used especially if the parasitic capacitances minimized in an on-chip design.

FIG. 11A illustrates an OOK data modulator. FIG. 11B depicts an envelope detector. FIG. 11C illustrates an oscilloscope screen shot for data transferred over the link.

This system employs concentric helical coils for wireless link in bioimplants as an alternative approach to the conventional options such as PSCs. It has been shown that this method provides a strong coupling between the coils (k=0.44) as well as high power transfer efficiency of 93.6%. It has been also shown that the coupling between the coils and power transfer efficiency did not change more than 19.9% and 9%, respectively, which result in a resilient and stable link. Furthermore, by decreasing the electromagnetic power density over living tissues as well as moving the wireless module to the less sensitive parts in the body, the higher amount of power could be safely delivered to the implant. For data transfer, a rate of 100 Kbps has obtained using 20 MHz carrier frequency for the backtelemetry link.

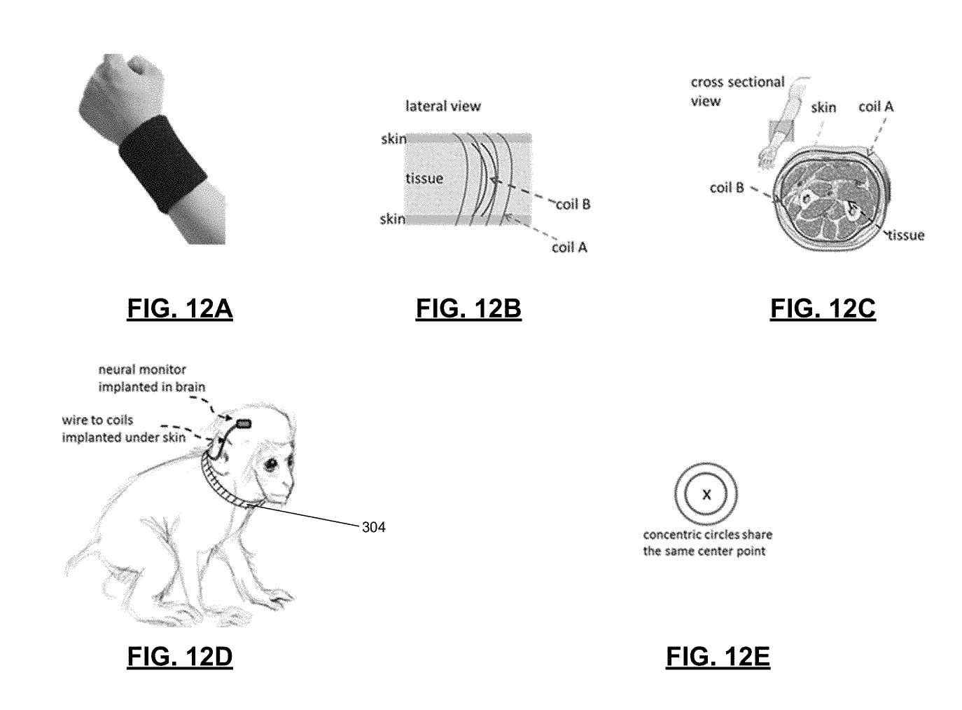

FIG. 12A illustrates an example concentric helical coil in a wearable bracelet. For example, as shown in FIG. 12B and FIG. 12C, for human use, a cardiac monitor may be implanted in the wrist, and the human may wear the concentric helical coil as a wearable bracelet with an embedded helical coil (coil A). Coil B is implanted beneath skin under bracelet for power/data telemetry.

FIG. 12D illustrates an example collar for animal research. In neural activity studies, a wearable animal collar includes an embedded helical coil (coil A) with coil B beneath skin under collar. Regardless of where the wireless power and data telemetry coils are positioned on a body, coil A is embedded within the cloth, leather, etc. of a wearable accessory and coil B is implanted just beneath the skin. The two coils are in a concentric formation as shown in FIG. 12E.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

What is claimed is:

1. A biotelemetry system comprising:
an implanted helical coil including at least one helical coil;
a wearable external helical coil configured to wirelessly transmit data to and receive data from the implanted helical coil, wherein the wearable external helical coil is further configured to wirelessly transmit power to the implanted helical coil, wherein the wearable external helical coil and the implanted helical coil are concentric, and wherein the wearable external helical coil includes at least one helical coil; and
an implanted electronic device connected to the implanted helical coil using a wire.

2. The biotelemetry system of claim 1 wherein the implanted helical coil includes:
a power regulator configured to deliver wirelessly transmitted power from the wearable external helical coil to the implanted electronic device via the wire.

3. The biotelemetry system of claim 1 wherein the implanted helical coil includes:
a data modulator configured to transmit data wirelessly from the implanted helical coil to the wearable external helical coil.

4. The biotelemetry system of claim 1 wherein the wearable external helical coil is connected to a battery and a storage device configured to store data transmitted from the implanted electronic device.

5. The biotelemetry system of claim 4 wherein the storage device is included in a computing system configured to record and process data, wherein the data received from the implanted electronic device represents biological information.

6. The biotelemetry system of claim 5 wherein the biological information indicates brain activity.

7. The biotelemetry system of claim 5 wherein the computing system communicates with a network configured to store the biological information, and wherein the biological information can be accessed remotely.

8. The biotelemetry system of claim 1 wherein the implanted electronic device is a health monitoring system, a disorder treatment system, or a prosthetic control system.

9. The biotelemetry system of claim 1 wherein the implanted helical coil and the wearable external helical coil each include: a resistor, a capacitor, and an inductor.

10. The biotelemetry system of claim 1 wherein the wearable external helical coil is configured to be worn around a neck or a wrist.
